(19) United States
(12) Patent Application Publication
(10) Pub. No.: US 2011/0137181 A1
(43) Pub. Date: Jun. 9, 2011

(54) Heart pulse detector

(75) Inventors: Shyuh Der Lin, Hsinchu city (TW); Yu Tang Lin, Hsinchu city (TW)

(73) Assignee: Holylite Microelectronics Corp., Hsinchu city (TW)

(21) Appl. No.: 12/654,012

(22) Filed: Dec. 8, 2009

(51) Int. Cl. A61B 5/024

(52) U.S. Cl. 600/479

(57) ABSTRACT

A heart pulse detector includes a light emitting diode for emitting a short pulse light toward body tissue and a photo sensor for receiving light signal modulated by blood pulse flow through body tissue. The output current of photo sensor is charging a capacitor during pulse light and is discharging the same capacitor during no pulse light for same period of pulse light. The ambient light signal is then cancelled out from the detected light signal. The light current signal is transferred to a voltage signal after sample and hold procession. Finally heart pulse signal is detected out from the voltage signal without the interference of ambient light.

### BACKGROUND OF THE INVENTION

1. Field of the Invention

The present invention relates to a heart pulse detector, and more particularly to a heart pulse detector which detects a user's heart pulse signal via a photo sensor to detect the light signal variation from the blood flow of the body tissue of user.

2. Description of the Related Art

There are various methods to detect the heart pulse signal of human body. Optical heart pulse detector is one of conventional device which comprises a light emitter and a photo sensor to detect the light signal variation from the blood flow of the tissue of human body and extract out the pulse signal of human body. To suppress the background ambient light signal from the signal detected by photo sensor is important to the performance of optical heart pulse detector. The asynchronous ambient light cancellation circuit is used in a heart rate measurement system of U.S. Pat. No. 4,258,719, to Lanny L. Lewyn. This circuit consists of a storage capacitor coupled between the gate and the source electrodes of a split-drain FET to store charge of equivalent ambient light current during intervals between IR (infra-red) carrier pulses and cancel the ambient light current from the output current of photodiode during an IR pulse. Since the charge stored by the capacitor is averaged out by integrating the current for a long time period as compared to the short time duty of current cancellation of an IR pulse, so the ambient light current will not be cancelled perfectly as ambient light intensity has a variation time to time.

### OBJECTS OF THE INVENTION

It is therefore an object of the invention to provide a heart pulse detector with improved ambient light cancellation circuit.

### DISCLOSURE OF THE INVENTION

A first aspect of the present invention teaches a heart pulse detector for detecting the heart pulse signal from blood pulse signal of human body, including the following parts: Timing generation means for providing periodically a first pulse with period in the range between 0.5 milliseconds to 20 milliseconds, and a second pulse with active interval of duration t2 in the range between 20 microseconds to 200 microseconds during non-active interval of the first pulse, and a third pulse with active interval of duration t2 during non-active interval of the first pulse and the start of active interval of the third pulse is right after the end of active interval of the second pulse, and a fourth pulse with active interval of duration t4 in the range between 20 microseconds to 200 microseconds during non-active interval of the first pulse and non-active interval of the second pulse and the start of active interval of the fourth pulse is after the end of active interval of the third pulse; A light emitting diode for emitting a pulsed light toward body tissue during the active intervals of the second pulse; A photo sensor for providing a sensor current signal in response to a received light signal which sourced from the pulsed light and ambient light and modulated by blood pulse flowing through body tissue; A first capacitor; A first switch connected to the two terminals of the first capacitor for discharging the first capacitor during the active intervals of the first pulse to a zero charge; Signal detection means for charging charge corresponding to the sensor current signal into the first capacitor during the active intervals of the second pulse and for discharging charge corresponding to the sensor current signal from the first capacitor during the active intervals of the third pulse to cancel the ambient light signal related charge previously stored in the first capacitor and for providing a first voltage signal corresponding to the blood pulse signal related charge stored in the first capacitor after the ambient light signal is cancelled; A second capacitor; Signal sampling means for storing the first voltage signal on the second capacitor during the active intervals of the fourth pulse and for providing a second voltage signal proportional to the voltage containing blood pulse signal stored on the second capacitor; Means for providing a heart pulse signal in response to blood pulse signal contained in the second voltage signal to the output of the heart pulse detector; A power regulator for providing a constant voltage source to the circuits of the heart pulse detector.

The signal detection means of the heart pulse detector including the following parts: A first operational amplifier having negative input terminal connected to a voltage reference source; A first p-channel field-effect transistor having source terminal connected to the constant voltage source and gate terminal connected to the output terminal of the first operational amplifier and drain terminal connected to the positive input terminal of the first operational amplifier and the output of the photo sensor to provide a first current reference voltage corresponding to the sensor current to the output terminal of the first operational amplifier; A second p-channel field-effect transistor having source terminal connected to the constant voltage source and gate terminal connected to the output terminal of the first operational amplifier to provide a first reference current proportional to the sensor current at a fixed ratio R to be output from drain terminal; A third p-channel field-effect transistor having source terminal connected to the constant voltage source and gate terminal connected to the output terminal of the first operational amplifier to provide a second reference current proportional to the sensor current at the fixed ratio R to be output from drain terminal; A first n-channel field-effect transistor having source terminal connected to the ground of system power supply and gate terminal and drain terminal both connected to the drain terminal of the second p-channel field-effect transistor to provide a second current reference voltage corresponding to the first reference current at drain terminal; A second n-channel field-effect transistor having source terminal connected to the ground of system power supply and gate terminal connected to the drain terminal of the first n-channel field-effect transistor to provide a third reference current corresponding to the first reference current at a ratio of one to be output from drain terminal; A second operational amplifier having positive input terminal connected to the voltage reference source and output terminal connected to one terminal of the first capacitor and negative input terminal connected to the other terminal of the first capacitor to charge the charge of the second reference current into the first capacitor through a second switch connected between the drain terminal of the third p-channel field-effect transistor and negative input terminal during the active intervals of the second pulse, and to discharge the charge of the third reference current from the first capacitor through a third switch connected between the drain terminal of the second n-channel field-effect transistor and negative input terminal during the active intervals of the third pulse, and to provide the first voltage signal at output terminal.

### BRIEF DESCRIPTION OF THE DRAWINGS

The foregoing and other advantages of the invention will be more fully understood with reference to the description of the best embodiment and the drawing wherein:

FIG. 1 is a block diagram of the present invention.

FIG. 2 is a block diagram of timing generator of the present invention.

FIG. 3 is a timing diagram of the system control signals of the present invention.

FIG. 4 is a diagram of LED driver of the present invention.

FIG. 5 is a diagram of photo emitter and sensor of the present invention

FIG. 6 is a block diagram of signal detector of the present invention.

### DESCRIPTION OF THE PREFERRED EMBODIMENTS

The foregoing and other advantages of the invention will be more fully understood with reference to the description of the best embodiment and the drawing as the following description.

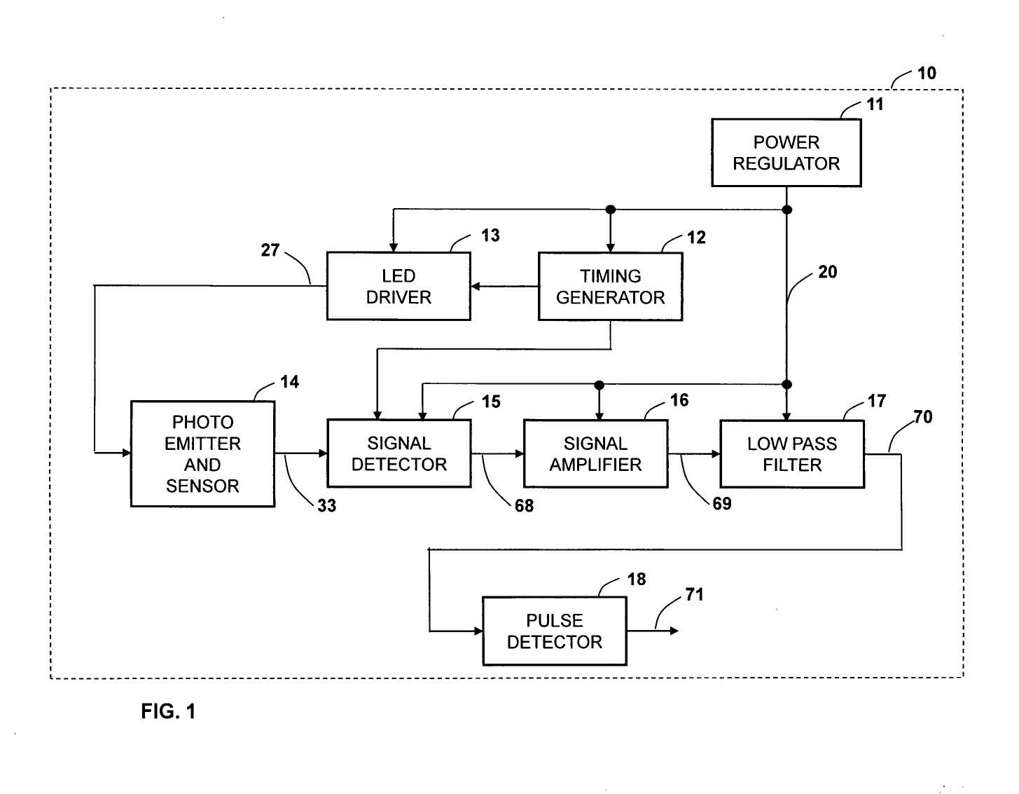

The preferred embodiment of present invention is illustrated in FIGS. 1-6.

As shown in FIG. 1, a heart pulse detector 10 comprises a power regulator 11 provide a constant voltage source 20 to the circuits in the heart pulse detector. A timing generator 12 generates control signals to LED driver 13 and signal detector 15. A photo emitter and sensor 14 receives the driving signal 27 output from LED driver 13 and outputs photo signal 33 to signal detector 15. A signal amplifier 16 follows signal detector 15 to amplify the detected signal 68 and the high frequency signal of output signal 69 of the signal amplifier 16 is filtered out by a low pass filter 17. A pulse detector 18 is placed on the last stage to detect the heart pulse signal 71 from the filtered signal 70.

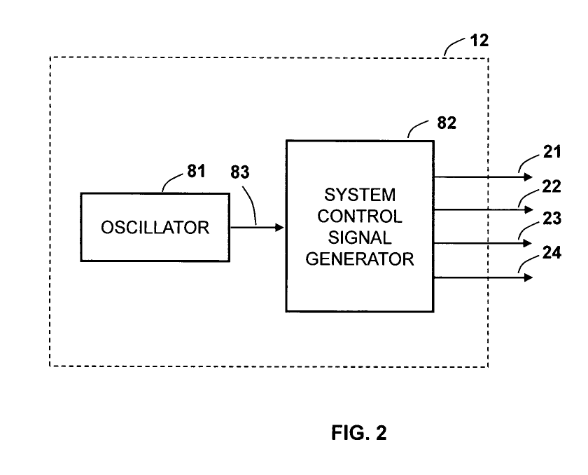

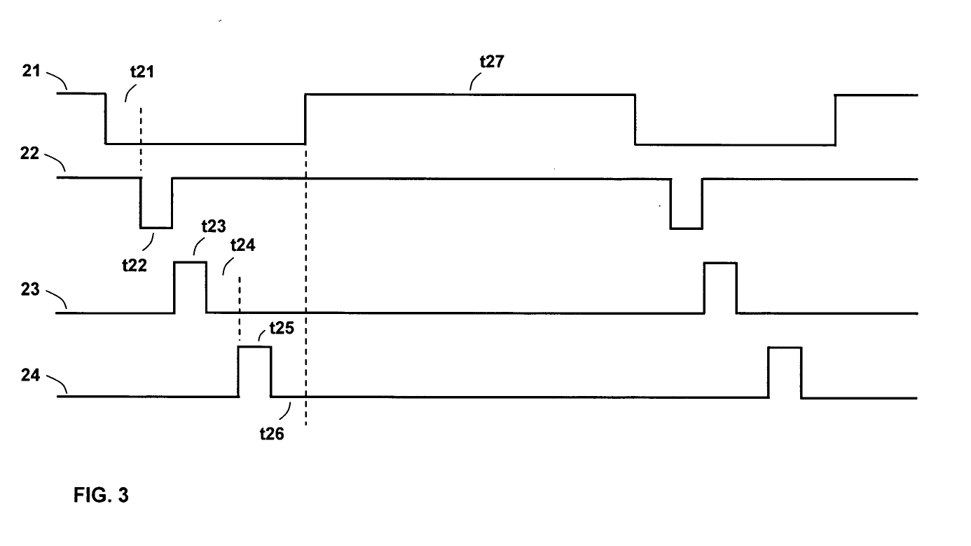

Referring to FIG. 2, a timing generator circuitry 12 includes an oscillator 81 and a system control signal generator 82. The oscillator 81 generate a constant frequency clock signal 83 for the system control signal generator to generate four system control signals which include a first control signal 21, a second control signal 22, a third control signal 23, and a fourth control signal 24. The timing sequences of the four system control signals are shown in FIG. 3. The four system control signals are periodical pulsed signals with same period, but have different duty of high level signal and low level signal. The timing phases between the four system control signals are fixed.

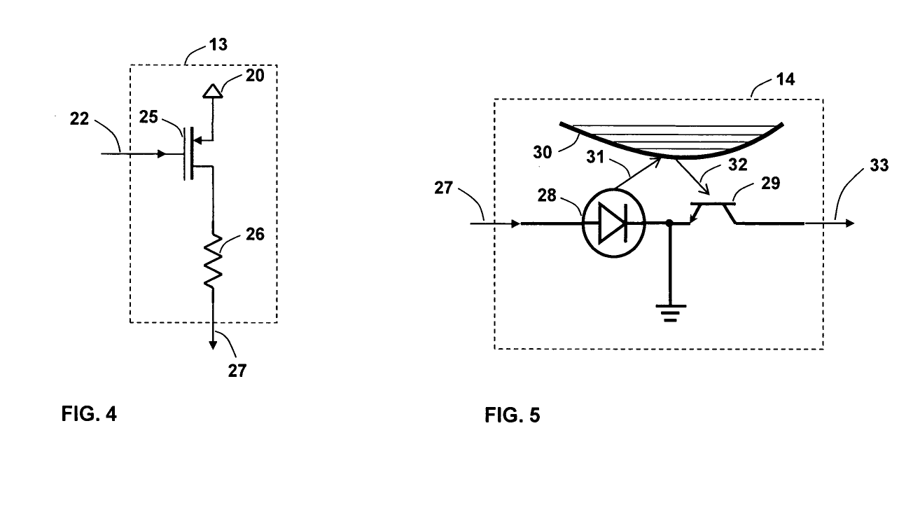

The detail circuits of the LED driver 13 are shown in FIG. 4. The gate terminal of a PMOS FET 25 is connected to the second control signal 22. The PMOS FET 25 will be turned on when the second control signal 22 is at low level and will be turned off when the second control signal 22 is at high level. The source terminal of the PMOS FET 25 is connected to the constant voltage source 20 and the drain terminal of the PMOS FET 25 is connected to one terminal of a resister 26. The driving signal 27 output from the LED driver 13 is connected to the other terminal of the resister 26.

As shown in FIG. 5, a light emitting diode (LED) 28 is used as the photo emitter and a phototransistor 29 is used as the photo sensor of the heart pulse detector 10 for example. The anode terminal of the LED 28 is connected to the input of the photo emitter and sensor 14 and receives the driving signal 27. The cathode terminal of the LED 28 is connected to the ground of the power supply of the heart pulse detector 10. When the PMOS FET 25 is turned on, the driving signal 27 output from the LED driver 13 will provide a constant voltage and a constant current to the LED 28, and a light beam 31 with constant intensity is emitted out from the LED 28 and light toward a finger 30 of human body. The photo signal 33 is output to the signal detector 15 from the collector terminal of the phototransistor 29 when a reflected light beam 32 is received by the phototransistor 29. The photo signal 33 is a photo current generated by phototransistor 29 responds to the intensity of the reflected light beam 32. The intensity of the reflected light beam 32 is related to the light beam 31 emitted from the LED 28 and the ambient light (not shown) both are modulated by blood pulse flowing through the tissue of the finger 30. When the second control signal 22 is at high level, the light beam 31 does not emitted from the LED 28, the photo signal 33 responds to the ambient light only.

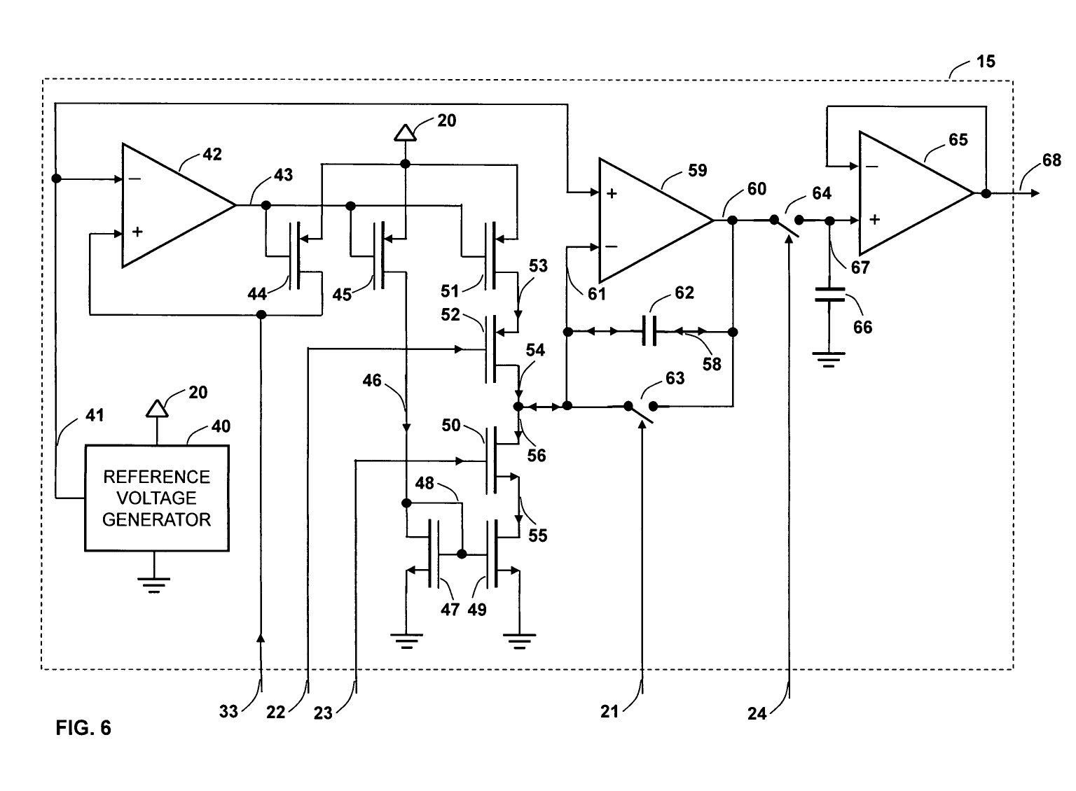

The detail circuits of the signal detector 15 are shown in FIG. 6, the photo signal 33 goes into the signal detector 15 from the drain terminal of a PMOS FET 44. The source terminal of the PMOS FET 44 is connected to the constant voltage source 20, and the gate terminal of the PMOS FET 44 is connected to the output terminal 43 of an operational amplifier 42. The negative input terminal of the operational amplifier 42 is connected to a reference voltage source 41 which is the output of a reference voltage generator 40. The positive input terminal of the operational amplifier 42 is connected to the drain terminal of the PMOS FET 44 to make a feedback loop for the operational amplifier 42. The voltage level of the reference voltage source 41 is designed to be a fixed ratio to the voltage level of the constant voltage source 20. A half voltage level of the constant voltage source 20 is the preferred value for the voltage level of the reference voltage source 41. The voltage at the drain terminal of the PMOS FET 44 will follow the voltage of the reference voltage source 41, so that the voltage between the source terminal and the drain terminal of the PMOS FET 44 is a constant voltage. Since the voltage signal at the output terminal 43 of the operational amplifier 42 responds to the drain current signal of the PMOS FET 44, so that the voltage signal at the output terminal 43 of the operational amplifier 42 will respond to the photo signal 33. The gate terminal of a PMOS FET 45 and the gate terminal of a PMOS FET 51 are connected to the output terminal 43 of the operational amplifier 42. The source terminal of the PMOS FET 45 and the source terminal of the PMOS FET 51 are connected to the constant voltage source 20. The drain terminal of the PMOS FET 45 is connected to the drain terminal of a NMOS FET 47. The gate terminal of the NMOS FET 47 and the gate terminal of a NMOS FET 49 are connected to the drain terminal of the NMOS FET 47 at node 48. The source terminal of the NMOS FET 47 and the source terminal of the NMOS FET 49 are connected to the ground of the power supply of the heart pulse detector 10. The drain terminal of the NMOS FET 49 is connected to the source terminal of a NMOS FET 50, and the drain terminal of the PMOS FET 51 is connected to the source terminal of a PMOS FET 52. The drain terminal of the PMOS FET 52 and the drain terminal of the NMOS FET 50 are connected to the negative input terminal 61 of an operational amplifier 59. The positive input terminal of the operational amplifier 59 is connected to the reference voltage source 41. One terminal of a capacitor 62 and one terminal of a switch 63 are connected to the negative input terminal 61 of the operational amplifier 59. The other terminal of the capacitor 62 and the other terminal of the switch 63 are connected to the output terminal 60 of the operational amplifier 59. One terminal of a capacitor 66 and one terminal of a switch 64 are connected to the positive input terminal 67 of an operational amplifier 65. The other terminal of the switch 64 is connected to the output terminal 60 of the operational amplifier 59, and the other terminal of the capacitor 66 is connected to the ground of the power supply of the heart pulse detector 10. The output terminal of the operational amplifier 65 is connected to the negative input terminal of the operational amplifier 65, so that the function of the operational amplifier 65 is a unit gain buffer. The first control signal 21 is input to the control terminal of the switch 63, and the fourth control signal 24 is input to the control terminal of the switch 64. The second control signal 22 is input to the gate terminal of the PMOS FET 52, and the third control signal 23 is input to the gate terminal of the NMOS FET 50.

When the first control signal 21 is at high level, the switch 63 is turned on, and the charge original stored in the capacitor 62 is discharged to zero. The second control signal 22 will change to low level from high level at a fixed short time period t21 after the first control signal 21 changes to low level from high level. When the second control signal 22 is at low level, the PMOS FET 52 will be turned on, and a current 54 will output from the drain terminal of the PMOS FET 52. Since the first control signal 21 and the third control signal 23 are both at low level, so that the switch 63 and the NMOS FET 50 are turned off, then a current 58 flow through the capacitor 62 is equivalent to the current 54. A current 53 output from the drain terminal of the PMOS FET 51 is proportional to the photo signal 33 at a fixed ratio between the size of the gate of the PMOS FET 51 and the PMOS FET 44. The preferred ratio is one to four. The gate size of the PMOS FET 51 is one quarter of the gate size of the PMOS FET 44. The gate size of the PMOS FET 45 is same as the gate size of the PMOS FET 51, and a current 46 output from the drain terminal of the PMOS FET 45 has the same value of the current 53. Since the current 46 flow into the drain terminal of the NMOS FET 47, then the voltage signal at the node 48 is correspond to the current 46. The gate size of the NMOS FET 49 is same as the gate size of the NMOS FET 47, by the control of the voltage signal at the node 48, a current 55 equal to the current 46 will be draw into the drain terminal of the NMOS FET 49. Since the current 54 is equivalent to the current 53, so that the current 58 is proportional to the photo signal 33 at a ratio of one to four. The charge being charged into the capacitor 62 by the current 58 contents the blood pulse signal related to the light beam 31 and the ambient light during this period.

Being at low level for a short time period t22, the second control signal 22 changes to high level and the third control signal 23 will changes to high level from low level at the same time, so that the PMOS FET 52 is turned off and the NMOS FET 50 is turned on. The current 54 is cut to zero and a current 56 is drawn into the drain terminal of the NMOS FET 50. Then the current 58 is changed from the current 54 to the current 56. Since the current 56 is equivalent to the current 55 which is drawn into the drain terminal of the NMOS FET 49, so that the current 58 is also proportional to the photo signal 33 at a ratio of one to four. Since the LED 28 is turned off and the direction of the flow of the current 58 through the capacitor 62 is reversed, so that the charge being discharged out from the capacitor 62 by the current 58 contains the blood pulse signal related to the ambient light only during this period.

Being at high level for a short time period t23, the third control signal 23 changes to low level, so that the NMOS FET 50 is turned off and the current 56 is cut to zero. Since the current 58 flows through the capacitor 62 is zero, the charge Qc62 remained in the capacitor 62 is kept unchanged. The short time period t22 and the short time period t23 have same time period. The preferred time period of the short time period t22 and the short time period t23 is 62 microseconds. Because the time period is short, the ambient light signal in the photo signal 33 at the short time period t23 is close to the ambient light signal in the photo signal 33 at the short time period t22, so that the ambient light signal charged into the capacitor 62 at the short time period t22 is discharged out from the capacitor 62 at the short time period t23. The remained charge Qc62 in the capacitor 62 contains the blood pulse signal related to the light beam 31 only, since the ambient light signal is cancelled out. The voltage signal at the output terminal 60 of the operational amplifier 59 becomes a DC reference voltage plus the voltage across the capacitor 62 which is related to the blood pulse signal.

The fourth control signal 24 will changes to high level from low level after a short time period t24 when the third control signal 23 changes to low level from high level. The switch 64 is turned on when the fourth control signal 24 is at high level. The voltage signal at the output terminal 60 of the operational amplifier 59 is transferred to the positive input terminal 67 of the operational amplifier 65 and is stored on the capacitor 66. The fourth control signal 24 will stay at high level for a short time period t25, then changes to low level to turn off the switch 64. The voltage signal 68 at the output terminal of the operational amplifier 65 is a sampled and held signal of blood pulse signal which is related to the light beam 31 only. The first control signal 21 is changed to high level from low level after a short time period t26 when the fourth control signal 24 changes to low level from high level. The switch 63 is turned on, and the charge in capacitor 62 is discharged to zero. The first control signal 21 stays at high level for a time period t27, then changes to low level to turn off the switch 63 and start the signal detection of next operation cycle. The prefer time period of an operation is 2 milliseconds, and the sampling frequency of the detected blood pulse signal is 500 Hz.

The detected signal 68 is transfer to the signal amplifier 16 by an AC coupling method, so that only the blood pulse signal is amplified for later process. The amplified signal 69 output from the signal amplifier 16 is transfer to the low pass filter 17. Since the maximum frequency of blood pulse signal is 4 Hz, this frequency is far lower than the sampling frequency of the detected blood pulse signal. A first order low pass filter is good enough to output a smoothed blood pulse signal. Finally the filtered signal 70 output from the low pass filter 17 is transfer to the pulse detector 18. The circuit of the pulse detector can be a voltage comparator with a reference voltage as the threshold of pulse signal. When the voltage of the incoming filtered signal 70 is higher than the threshold voltage, the heart pulse signal 71 is detected and output from the pulse detector 18.

The heart pulse detector of the present invention can achieve a good result in the cancellation of the ambient light signal from the sensed photo blood pulse signal and has great help to the detection of heart pulse signal.

Although specific embodiments of the invention have been disclosed, it will be understood by those having skill in the art that minor changes can be made to the form and details of the specific embodiments disclosed herein, without departing from the scope of the invention. The embodiments presented above are for purposes of example only and are not to be taken to limit the scope of the appended claims.

What is claimed is:

1. A heart pulse detector for detecting the heart pulse signal from blood pulse signal of human body, comprising:
timing generation means for providing periodically a first pulse with period in the range between 0.5 milliseconds to 20 milliseconds, and a second pulse with active interval of duration t2 in the range between 20 microseconds to 200 microseconds during non-active interval of said first pulse, and a third pulse with active interval of duration t2 during non-active interval of said first pulse and the start of active interval of said third pulse is right after the end of active interval of said second pulse, and a fourth pulse with active interval of duration t4 in the range between 20 microseconds to 200 microseconds during non-active interval of said first pulse and non-active interval of said second pulse and the start of active interval of said fourth pulse is after the end of active interval of said third pulse;
a light emitting diode for emitting a pulsed light toward body tissue during the active intervals of said second pulse;
a photo sensor for providing a sensor current signal in response to a received light signal which sourced from said pulsed light and ambient light and modulated by blood pulse flowing through body tissue;
a first capacitor;
a first switch connected to the two terminals of said first capacitor for discharging said first capacitor during the active intervals of said first pulse to a zero charge;
signal detection means for charging charge corresponding to said sensor current signal into said first capacitor during the active intervals of said second pulse and for discharging charge corresponding to said sensor current signal from said first capacitor during the active intervals of said third pulse to cancel the ambient light signal related charge previously stored in said first capacitor and for providing a first voltage signal corresponding to the blood pulse signal related charge stored in said first capacitor after the ambient light signal is cancelled;
a second capacitor;
signal sampling means for storing said first voltage signal on said second capacitor during the active intervals of said fourth pulse and for providing a second voltage signal proportional to the voltage containing blood pulse signal stored on said second capacitor;
means for providing a heart pulse signal in response to blood pulse signal contained in said second voltage signal to the output of said heart pulse detector;
a power regulator for providing a constant voltage source to the circuits of said heart pulse detector.

2. A heart pulse detector as claimed in claim 1, wherein said signal detection means is comprised of
a first operational amplifier having negative input terminal connected to a voltage reference source;
a first p-channel field-effect transistor having source terminal connected to said constant voltage source and gate terminal connected to the output terminal of said first operational amplifier and drain terminal connected to the positive input terminal of said first operational amplifier and the output of said photo sensor to provide a first current reference voltage corresponding to said sensor current to the output terminal of said first operational amplifier;
a second p-channel field-effect transistor having source terminal connected to said constant voltage source and gate terminal connected to the output terminal of said first operational amplifier to provide a first reference current proportional to said sensor current at a fixed ratio R to be output from drain terminal;
a third p-channel field-effect transistor having source terminal connected to said constant voltage source and gate terminal connected to the output terminal of said first operational amplifier to provide a second reference current proportional to said sensor current at said fixed ratio R to be output from drain terminal;
a first n-channel field-effect transistor having source terminal connected to the ground of system power supply and gate terminal and drain terminal both connected to the drain terminal of said second p-channel field-effect transistor to provide a second current reference voltage corresponding to said first reference current at drain terminal;
a second n-channel field-effect transistor having source terminal connected to the ground of system power supply and gate terminal connected to the drain terminal of said first n-channel field-effect transistor to provide a third reference current corresponding to said first reference current at a ratio of one to be output from drain terminal;
a second operational amplifier having positive input terminal connected to said voltage reference source and output terminal connected to one terminal of said first capacitor and negative input terminal connected to the other terminal of said first capacitor to charge the charge of said second reference current into said first capacitor through a second switch connected between the drain terminal of said third p-channel field-effect transistor and negative input terminal during the active intervals of said second pulse, and to discharge the charge of said third reference current from said first capacitor through a third switch connected between the drain terminal of said second n-channel field-effect transistor and negative input terminal during the active intervals of said third pulse, and to provide said first voltage signal at output terminal.
